(19) United States
(12) Patent Application Publication
(10) Pub. No.: US 2010/0249842 A1
(43) Pub. Date: Sep. 30, 2010

(54) SPINOUS PROCESS CROSS-LINK

(75) Inventor: Hamid R. Mir, Santa Monica, CA (US)

Correspondence Address: PEARNE & GORDON LLP, 1801 EAST 9TH STREET, SUITE 1200, CLEVELAND, OH, 44114-3108, US

(73) Assignee: Dr. Hamid R. Mir, Santa Monica, CA (US)

(21) Appl. No.: 12/415,010

(22) Filed: Mar. 31, 2009

(51) Int. Cl. A61B 17/70

(52) U.S. Cl. 606/250

(57) ABSTRACT

The present invention relates to a prosthetic spinous process cross-link for the replacement of a posterior vertebral element. The vertebral element may include a natural lamina extending from a pair of natural pedicles and a natural spinous process extending from the lamina. The prosthetic spinous process cross-link may include a first pair and a second pair of screws attached to a vertebral body and a first rod and a second rod, the first rod extending between the first pair of screws and the second rod extending between the second pair of screws. A prosthetic lamina attached to the rods is also provided, along with a prosthetic spinous process extending from the prosthetic lamina.

### FIELD OF THE INVENTION

The present invention relates generally to surgical implants, and more particularly to implants that replace posterior vertebral elements such as a natural lamina and a natural spinous process.

### BACKGROUND OF THE INVENTION

Implantable surgical devices are known and used in many different applications, including spinal surgery. A prosthetic device may be attached to a posterior section of a vertebra to mimic a natural vertebral element. In one example, an implantable prosthetic device is attached to the posterior vertebra by screws and designed to replace the natural lamina, the natural spinous process, all four natural facets, and may also replace the natural transverse processes. However, during a lumbar laminectomy, only the lamina and spinous process are removed, as opposed to the complete spinal vertebra. Thus, there is a continuing need for a prosthetic device that replaces only the lamina and spinous process.

### BRIEF SUMMARY OF THE INVENTION

The following presents a simplified summary of the invention in order to provide a basic understanding of some example aspects of the invention. This summary is not an extensive overview of the invention. Moreover, this summary is not intended to identify critical elements of the invention nor delineate the scope of the invention. The sole purpose of the summary is to present some concepts of the invention in simplified form as a prelude to the more detailed description that is presented later.

In accordance with one aspect of the present invention, a prosthetic spinous process cross-link, hereinafter referred to as “implant”, for the replacement of a posterior vertebral element is provided. Posterior vertebral element is defined as posterior spinous process and the lamina. The vertebral element includes a natural lamina extending from a pair of natural pedicles and a natural spinous process extending from the lamina. The implant includes a first pair and a second pair of screws attached to a vertebral body and a first rod and a second rod, the first rod extending between the first pair of screws and the second rod extending between the second pair of screws. A prosthetic lamina attached to the rods is also provided, along with a prosthetic spinous process extending from the prosthetic lamina.

In accordance with another aspect of the present invention, a prosthetic spinous process cross-link, hereinafter referred to as “implant”, for the replacement of a posterior vertebral element is provided. Posterior vertebral element is defined as posterior spinous process and the lamina. The vertebral element includes a natural lamina extending from a pair of natural pedicles extending from the natural vertebral body, a natural lamina extending from the pair of pedicles, a pair of natural superior and inferior facets extending from the natural lamina, and a natural spinous process extending from the lamina. The implant includes a first pair and a second pair of C-shaped gripping portions that attach the implant to the vertebral body using a first rod and a second rod, the first rod extending between a first pair of screws and the second rod extending between a second pair of screws. A prosthetic lamina attached to the rods is also provided, along with a prosthetic spinous process extending from the prosthetic lamina.

In accordance with another aspect of the present invention, a prosthetic spinous process cross-link, hereinafter referred to as “implant”, is provided for the replacement of a posterior vertebral element. The vertebral element includes a natural lamina extending from a pair of natural pedicles and a natural spinous process extending from the lamina. The implant includes a mounting assembly attached to a vertebral body. The implant consists of a prosthetic lamina and prosthetic spinous process along with at least one aperture extending from the spinous process.

### BRIEF DESCRIPTION OF THE DRAWINGS

The foregoing and other aspects of the present invention will become apparent to those skilled in the art to which the present invention relates upon reading the following description with reference to the accompanying drawings, in which:

FIG. 1 illustrates a cross-sectional view of a lumbar vertebra;

FIG. 2 illustrates a cross-sectional view of a lumber vertebra along with attached muscles;

FIG. 3 illustrates a posterior view of the vertebral body following a lumbar laminectomy;

FIG. 4 illustrates a perspective view of a spinous process cross-link;

FIG. 5 illustrates a perspective view of a pedicle screw assembly;

FIG. 6 illustrates a side view of the spinous process cross-link of FIG. 4 that replaces the lamina and spinous process; and

FIG. 7 illustrates a posterior view of the spinous process cross-link of FIG. 5.

### DETAILED DESCRIPTION OF THE INVENTION.

Example embodiments that incorporate one or more aspects of the present invention are described and illustrated in the drawings. These illustrated examples are not intended to be a limitation on the present invention. For example, one or more aspects of the present invention can be utilized in other embodiments and even other types of devices. Moreover, certain terminology is used herein for convenience only and is not to be taken as a limitation on the present invention. Still further, in the drawings, the same reference numerals are employed for designating the same elements.

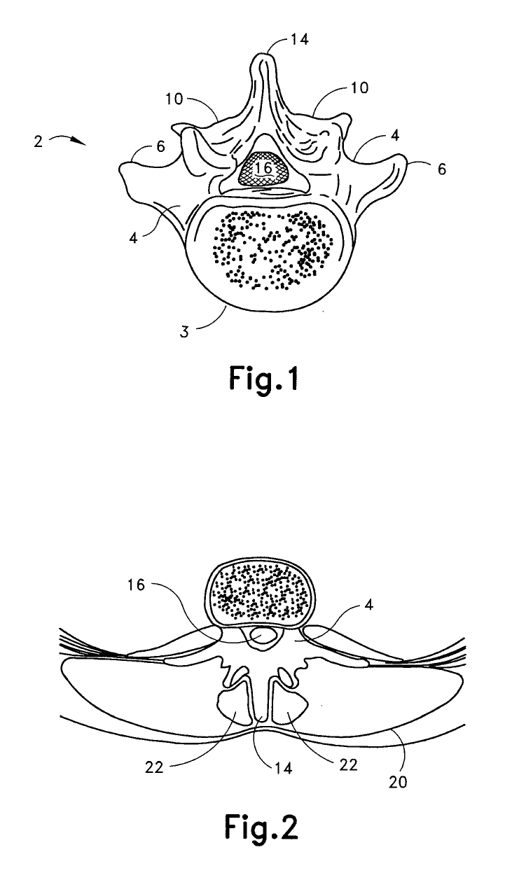

Turning to the shown example of FIG. 1, there is shown a cross-sectional view of a vertebral element 2. The vertebral element 2 comprises a pair of natural pedicles 4 extending from a vertebral body 3. A pair of transverse processes 6 extend from the natural pedicles 4. A natural lamina 10 also extends from the natural pedicles 4. A natural spinous process 14 is formed from the natural lamina 10. The natural spinous process 14 and natural lamina 10 surround a dura 16. As shown in FIG. 2, a lumbar fascia 20 covers a sheet of paraspinal muscles 22 in the back which extends from the natural spinous process 14. Paraspinal muscles 22 are located adjacent to the spine and pass over the lamina bilaterally.

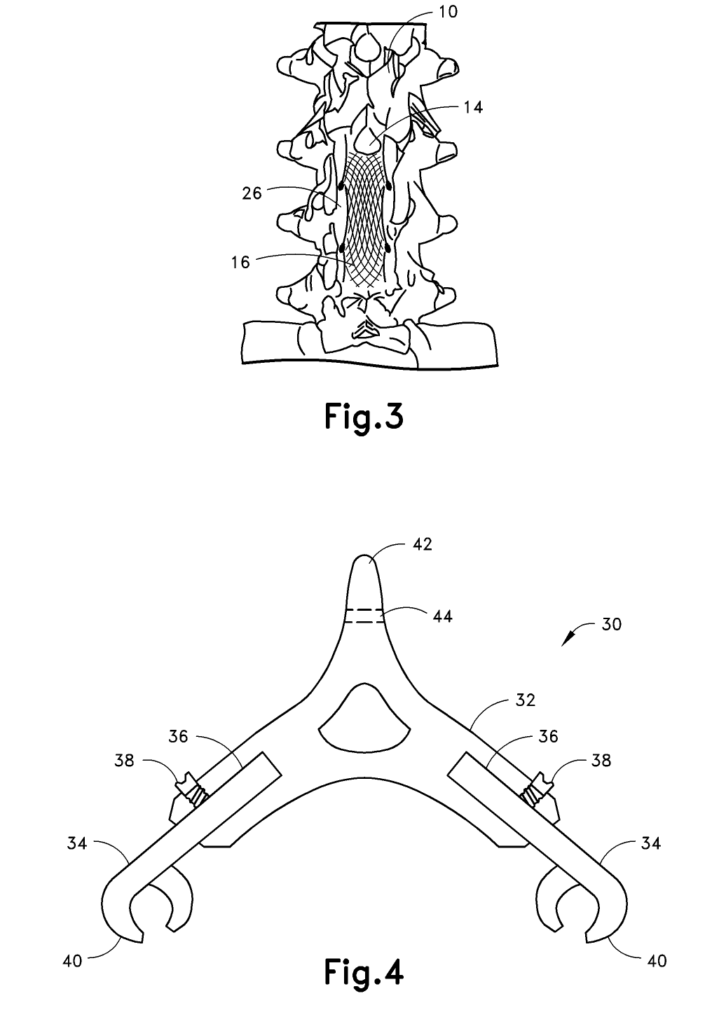

Referring now to FIG. 3, there is shown a vertebral element 2 after a lumbar laminectomy procedure. Compression of the dura 16 in the spine, also known as spinal stenosis, may affect the cervical, thoracic, or lumbar portions of the spine. One source of the compression may be due to the natural lamina 10 and natural spinous process 14 applying pressure to the dura 16. A lumbar laminectomy procedure alleviates the pressure by removing the natural lamina 10 and the natural spinous process 14. After an incision in a patient's skin, the lumbar fascia 20 and paraspinal muscles 22 are drawn away from the laminectomy site. Next, the natural lamina 10 and natural spinous process 14 are resected, exposing the dura 16 and alleviating any pressure. After the laminectomy a void 26 is created leaving both the dura 16 and spinal cord exposed. The void 26 may cause several problems including, but not limited to: 1) creation of deadspace allowing for a hematoma and seroma collection, a source for postoperative infection, and possible persistent compression of the dura 16. 2) As the natural spinous process 14 is removed, the re-attachment site for the lumbar fascia is lost. Therefore, the lumbar fascia 20 and paraspinal muscles 22 are unable to return to their natural positions. This often leads to a loss of muscle function and a source of back pain. A spinous process cross-link, hereinafter referred to as “implant”, 30 replaces the attachment site for the paraspinal muscles 22 by providing an attachment site at apertures 44 for the lumbar fascia 20. 3) The natural lamina 10 and natural spinous process 14 naturally encase and protect the dura 16. After a laminectomy, an abundance of scar tissue is formed following surgery. This makes it difficult to identify the location of the dura 16, making a subsequent surgical procedure dangerous with a risk of dural 16 injury during any revision surgery. The implant 30 provides protection and identification of the dura 16 in revision surgeries.

Referring now to FIG. 4, there is shown the implant 30 which is adapted to replace the natural lamina 10 and the natural spinous process 14 following the lumbar laminectomy procedure. The implant 30 comprises a cross-link body 32 and a pair of feet 34. One end of each foot 34 is inserted into a recess 36 within the cross-link body 32 and may be secured in place by screws 38. The screws 38 may be inserted into the cross-link body 32 to hold the feet 34 in place. The screws 38 allow for sliding of the feet 34 into the recess 36 with adjustment of the length. The final length of the feet 34 may ultimately be controlled by turning the screws 38. At the opposite end of the recess 36, each foot 34 may include a C-shaped gripping portion 40. The implant 30 is adapted to act as a prosthetic lamina and a prosthetic spinous process. The prosthetic lamina has an inverted U-shape with an elevated portion forming a prosthetic spinous process 42. The prosthetic spinous process 42 may include one or more apertures 44 extending through. The apertures 44 may comprise a number of different embodiments, such as a circular aperture, a plurality of circular apertures, one or more rectangular apertures, etc. The apertures allow for sutures to pass through following surgery. The sutures may assist in returning the paraspinal muscles 22 and lumbar fascia 20 and other tissues to their original positions. In one embodiment, the implant 30 may be made of titanium. Other materials, such as stainless steel mesh titanium, etc., are also contemplated. Similarly, the implant 30 may comprise varying sizes as well. For instance, the cross-link body 32 may be larger or wider to match the size of the vertebral body 3.

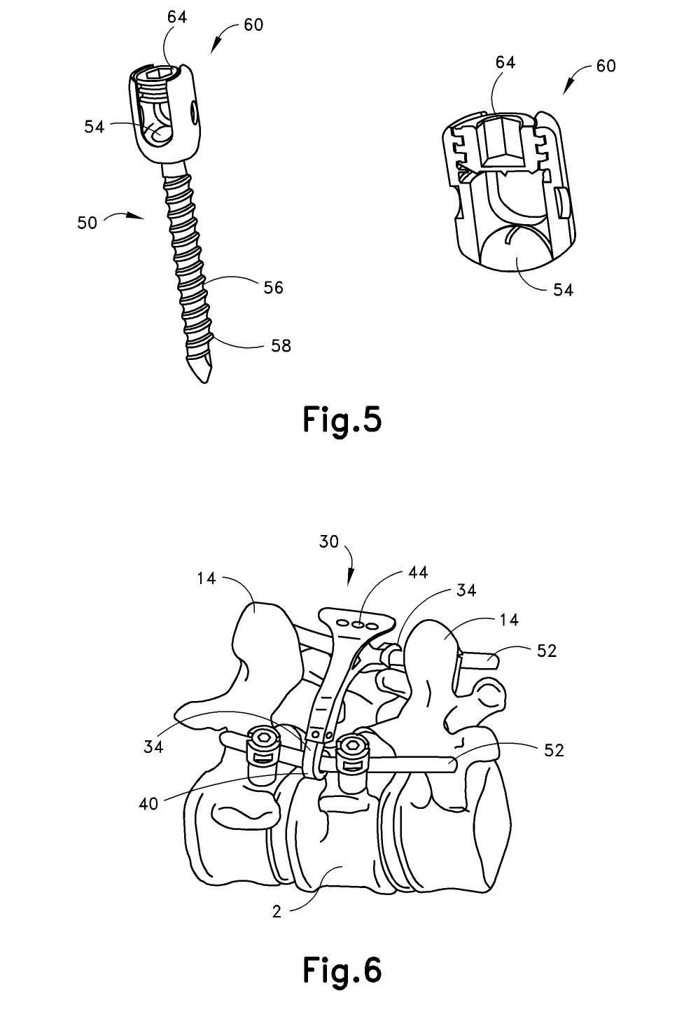

Referring to FIGS. 5 and 6, the implant 30 may be mounted to the vertebral body 3 by pedicle screws 50 and rods 52. Two pairs of pedicle screws 50 are provided with each pedicle screw 50 having a head portion 54 and a body portion 56. The body portion 56 of each screw has helical threads 58 on an exterior surface to screw into the vertebral body 3. A nut assembly 60 is also provided for receiving each pedicle screw 50 and for receiving the rod 52. As shown in FIG. 6, each pair of pedicle screws 50 is inserted into the nut assembly 60 and driven through a side of the vertebral body 3. Once the pedicle screws 50 are secured, the rods 52 may be inserted through an opening 62 in the nut assembly 60. The rods 52 are then held in place by a locking mechanism 64 which locks into the nut assembly 60. Each rod 52 extends between the pair of pedicle screws 50 and the rods 52 are substantially parallel to each other. Finally, each foot 34 from the implant 30 may be attached to one of the rods 52. The C-shaped gripping portion 40 of each foot 34 may be clamped onto the rods 52. The C-shaped gripping portion 40 and rod 52 connect to form a tight fit to limit any sliding of the implant 30. In the final position, the implant 30 is placed in the void 26 where the lumbar laminectomy procedure has occurred and is situated between two natural spinous processes 14.

The implant 30 may further act as a location for the placement of bone. Bone may be laid on top of the prosthesis, including on the cross-link body 32, on top of each foot 34, on top of the screws 38 and rods 52, etc. The prosthesis can be made from materials that can enhance or promote bony ongrowth. This may include materials including, but not limited to, hydroxyapatite, titanium mesh, etc. The bone placed on the prosthesis and the associated parts allows for regrowth of the bone and fortification of the fusion mass across the levels.

The mounting assembly and method described above is merely one of a number of potential mounting assemblies and methods. Various mounting assemblies, including, but not limited to the pedicle screw 50 and rod 52 assembly, are also contemplated. For instance, in one embodiment, the pedicle screws 50 alone may constitute the mounting assembly, as the implant 30 may be attached directly to the pedicle screws 50. In such an embodiment, the rods 52 may not be used and the implant feet 34 may be secured directly to the pedicle screws 50 as opposed to the rods 52. In yet another embodiment, each foot 34 may constitute a mounting assembly, as each foot 34 may have a screw portion instead of the C-shaped gripping portion 40. In such an embodiment, the screw portions may screw directly into the vertebral body 3, thus eliminating the need for both the pedicle screws 50 and rods 52. As shown in FIG. 6, the pedicle screws 50 may be inserted into more than one vertebral body 3 with the rods 52 extending therebetween. In a similar but different embodiment, a plurality of pedicle screws 50 may be inserted into a single vertebral body 3. Thus, rods 52 may still be used to extend between the pedicle screws 50 and allow for the C-shaped gripping portion 40 to be attached to the rods 52. In this instance, both the pedicle screws 50 and rods 52 may constitute the mounting assembly as the laminectomy may be performed without a spinal fusion. The implant 30 will then act to protect and identify the dura 16, but will not cause vertebral bodies to be fused together.

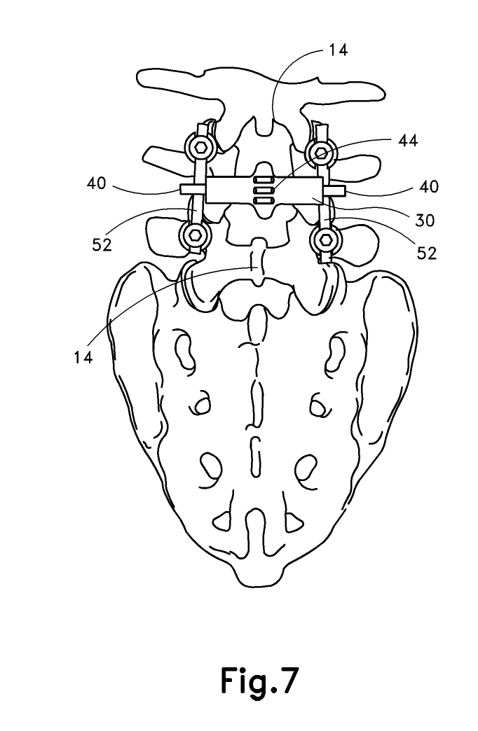

Referring now to FIG. 7, there is shown a side view of the Implant 30 attached to the vertebral body 3 after the lumbar laminectomy procedure. As previously mentioned, the prosthetic spinous process 42 may include one or more apertures 44 extending through. After the implant 30 is implanted on the vertebral body 3, the lumbar fascia 20 and paraspinal muscles 22 remain detached. The apertures 44 allow sutures to pass through for closure and reattachment of the lumbar fascia 20 and paraspinal muscles 22 following the lumbar laminectomy procedure. Once the lumbar fascia 20 are closed, the paraspinal muscles 22 may be brought back to their normal position adjacent to the cross-link body 32 and the prosthetic spinous process 42.

The invention has been described with reference to the example embodiments described above. Modifications and alterations will occur to others upon a reading and understanding of this specification. Examples embodiments incorporating one or more aspects of the invention are intended to include all such modifications and alterations insofar as they come within the scope of the appended claims.

What is claimed is:

1. A prosthetic spinous process cross-link for the replacement of a posterior vertebral element, wherein the vertebral element includes a natural lamina extending from a pair of natural pedicles and a natural spinous process extending from the natural lamina, the prosthetic spinous process cross-link including:
a first pair and a second pair of screws attached to a vertebral body;
a first rod and a second rod, the first rod extending between the first pair of screws and the second rod extending between the second pair of screws;
a prosthetic lamina attached to the rods; and
a prosthetic spinous process extending from the prosthetic lamina.

2. The prosthetic spinous process cross-link of claim 1 wherein the prosthetic spinous process includes an attachment site for the re-attachment of tissue.

3. The prosthetic spinous process cross-link of claim 2 wherein the attachment site includes at least one aperture extending through the prosthetic spinous process.

4. The prosthetic spinous process cross-link of claim 3 wherein the attachment site includes three apertures extending through the prosthetic spinous process.

5. The prosthetic spinous process cross-link of claim 1 wherein the prosthetic lamina includes a pair of feet, wherein each foot attaches to one of the rods.

6. The prosthetic spinous process cross-link of claim 1 wherein the prosthetic lamina is comprised of a material allowing for the reattachment and regrowth of bone.

7. A prosthetic spinous process cross-link for the replacement of a posterior vertebral element, wherein the vertebral element includes a pair of natural pedicles extending from the natural vertebral body, a natural lamina extending from the pair of pedicles, a pair of natural superior and inferior facets extending from the natural lamina, and a natural spinous process extending from the natural lamina, the prosthetic spinous process cross-link including:
a first pair and a second pair of screws attached to the vertebral body;
a first rod and a second rod, the first rod extending between the first pair of screws and the second rod extending between the second pair of screws;
a prosthetic lamina attached to the rods; and
a prosthetic spinous process extending from the prosthetic lamina.

8. The prosthetic spinous process cross-link of claim 7 wherein the prosthetic spinous process includes an attachment site for the re-attachment of tissue.

9. The prosthetic spinous process cross-link of claim 8 wherein the attachment site includes at least one aperture extending through the prosthetic spinous process.

10. The prosthetic spinous process cross-link of claim 9 wherein the attachment site includes three apertures extending through the prosthetic spinous process.

11. The prosthetic spinous process cross-link of claim 7 wherein the prosthetic lamina includes a pair of feet, wherein each foot attaches to one of the rods.

12. The prosthetic spinous process cross-link of claim 7 wherein the prosthetic lamina is comprised of a material allowing for the reattachment and regrowth of bone.

13. A prosthetic spinous process cross-link for the replacement of a posterior vertebral element, wherein the vertebral element includes a natural lamina extending from a pair of natural pedicles and a natural spinous process extending from the lamina, the prosthetic spinous process cross-link including:
a mounting assembly attached to a vertebral body;
a prosthetic lamina attached to the mounting assembly;
a prosthetic spinous process extending from the prosthetic lamina; and
at least one aperture extending through the spinous process.

14. The prosthetic spinous process cross-link of claim 13 wherein the mounting assembly includes a plurality of screws and a pair of rods, wherein each rod extends between a pair of screws.

15. The prosthetic spinous process cross-link of claim 14 wherein the prosthetic lamina includes a pair of feet, wherein each foot attaches to one of the rods.

16. The prosthetic spinous process cross-link of claim 13 wherein the at least one aperture serves as an attachment site for the re-attachment of tissue.

17. The prosthetic spinous process cross-link of claim 13 wherein the attachment site includes three apertures extending through the prosthetic spinous process.

18. The prosthetic spinous process cross-link of claim 17 wherein the three apertures serve as an attachment site for the re-attachment of tissue.

19. The prosthetic spinous process cross-link of claim 13 wherein the prosthetic lamina is comprised of a material allowing for the reattachment and regrowth of bone.
